(19) United States
(12) Patent Application Publication
(10) Pub. No.: US 2025/0253032 A1
(43) Pub. Date: Aug. 7, 2025

(54) DIGITAL THERAPEUTIC FOR TREATMENT OF PERINATAL MOOD DISORDERS

(71) Applicant: Curio Digital Therapeutics, Inc., Princeton, NJ (US)

(72) Inventors: Shailja DIXIT, Skillman, NJ (US); Sidhartha SHANKAR, Skillman, NJ (US)

(21) Appl. No.: 19/044,475

(22) Filed: Feb. 3, 2025

Related U.S. Application Data
(60) Provisional application No. 63/548,736, filed on Feb. 1, 2024.

(51) Int. Cl. G16H 20/70; A61K 45/06; A61P 25/24

(52) U.S. Cl. CPC G16H 20/70; A61K 45/06; A61P 25/24

(57) ABSTRACT

A method for treating perinatal mood disorders in women diagnosed with or at risk for the perinatal mood disorders. The method includes administering a digital therapeutic to the subject, the digital therapeutic configured to provide cognitive behavioral therapy content to the subject to treat the perinatal mood disorders, wherein the perinatal mood disorders are treated, thereby causing the subject to exhibit an improvement upon completion of the cognitive behavioral therapy content relative to a control subject that was not administered the digital therapeutic. In some embodiments, the digital therapeutic can be administered in conjunction with a pharmacotherapeutic.

### CROSS-REFERENCE TO RELATED APPLICATIONS

The present application claims priority to U.S. Provisional Patent Application No. 63/548,736, titled DIGITAL THERAPEUTIC FOR TREATMENT OF PERINATAL MOOD DISORDERS, filed Feb. 1, 2024, which is hereby incorporated by reference herein in its entirety.

### BACKGROUND

Perinatal mood disorders are a debilitating set of mental health conditions that affect one in seven women. Up to 85% of mothers have experienced postpartum blues within the first 10 days after giving birth, with a peak incidence occurring at the fifth day. However, postpartum depression (PPD) lasts longer and is a more serious condition than postpartum blues. Also, postpartum blues typically resolve on their own accord, while PPD does not and often requires more targeted treatment. Common symptoms of PPD include mood swings, mild elation, irritability, tearfulness, fatigue, and confusion.

Numerous studies have reported on the low rates of screening, diagnosis, and treatment of perinatal depression in medical settings. Each woman experiences PPD and its associated symptoms differently. Research has demonstrated that there are differences in symptoms for individual women in the perinatal period, including how early it starts, how severe the manifestations are, and how long it lasts. There are also varying rates of depression reported by race/ethnicity that may impact access to care and services received. It can be difficult for new mothers to care for themselves and/or their babies, and many women may not recognize the symptoms of PPD or be aware that effective treatments are available. Although postpartum depression affects between 6.5% and 12.9% of new mothers after childbirth, it often goes undetected and underdiagnosed. The main tools used to screen and identify patients with suspected PPD include both clinician-administered instruments (e.g., the Hamilton Rating Scale for Depression or HAMD) and validated patient reported outcome measures (PROMs), such as the Edinburgh Postnatal Depression Scale (EPDS), the Post-Partum Depression Scale (PPDS), and Patient Health Questionnaire-9 (PHQ9). Meta-analyses have validated the strong correlations between EPDS and PHQ9 and a postpartum depression diagnosis. Specifically, research shows the EPDS assessments have sensitivity and specificity rates as high as 74% and 97%, respectively, in the postpartum period.

Certain factors may increase the likelihood of depression during or after pregnancy, including history of depression or substance abuse, family history of mental illness, inadequate support from family and friends, anxiety about the fetus, problems with previous pregnancy or birth, marital or financial problems, young age (of mother or newborn). Tiredness, problems sleeping, stronger emotional reactions, hormonal changes, and changes in body weight normally occur during and after pregnancy, but these symptoms may also be signs of depression. It is natural for women to experience changes in their feelings and mood during pregnancy, including feeling more tired, irritable or worried. However, while mild mood changes during pregnancy are common, mood symptoms can sometimes become severe enough to require treatment by a health provider. Feelings of depression or anxiety persisting for a few weeks or that interfere with daily activities may require medical intervention. Depression and anxiety during pregnancy can worsen and continue into the postpartum period. Current treatment options, including Brexanolone and Zuranolone, are burdensome for subjects, are not cost-effective, and may not be appropriate for all subjects. For example, Brexanolone is administered as a 60-hour continuous intravenous infusion. As such, subjects face significant practical barriers to completing this treatment. As another example, Zuranolone has recently been approved for treating postpartum depression, but it may not be appropriate for all subjects and may not be cost-effective.

Studies show that many depressed mothers do not get proper treatment. Rather than seeking treatment, many mothers might instead attempt to cope with their condition. This self-treatment process can have long-lasting negative consequences for women though. Therefore, there is a need for systems that are able to assess individuals' states of mind and their risk for PPD in order to provide appropriate support to women, whether currently pregnant and post-pregnancy, that they are lacking under the current healthcare regime.

### SUMMARY

In some embodiments, there is provided a method for treating perinatal mood disorders in a subject diagnosed with or at risk for the perinatal mood disorders, the method comprising: administering a digital therapeutic to the subject, the digital therapeutic configured to provide cognitive behavioral therapy content to the subject to treat the perinatal mood disorders; wherein the perinatal mood disorders are treated, thereby causing the subject to exhibit an improvement of four or more points on the Edinburgh Postnatal Depression Scale (EPDS) upon completion of the cognitive behavioral therapy content relative to a control subject that was not administered the digital therapeutic.

In some embodiments, there is provided a method for treating perinatal mood disorders in a subject diagnosed with or at risk for the perinatal mood disorders, the method comprising: administering a digital therapeutic to the subject, the digital therapeutic configured to provide cognitive behavioral therapy content to the subject to treat the perinatal mood disorders; wherein the perinatal mood disorders are treated, thereby causing the subject to exhibit an Edinburgh Postnatal Depression Scale (EPDS) score of less than 13 upon completion of the cognitive behavioral therapy content relative to a control subject that was not administered the digital therapeutic.

In some embodiments, there is provided, a method for treating perinatal mood disorders in a subject diagnosed with or at risk for the perinatal mood disorders, the method comprising: administering a digital therapeutic to the subject, the digital therapeutic configured to provide cognitive behavioral therapy content to the subject to treat the perinatal mood disorders; wherein the perinatal mood disorders are treated, thereby causing the subject to exhibit an improved score on the Hamilton Rating Scale for Depression (HAMD) upon completion of the cognitive behavioral therapy content relative to a control subject that was not administered the digital therapeutic.

In one embodiment, the perinatal mood disorders comprise postpartum depression.

In one embodiment, the digital therapeutic comprises a plurality of modules.

In one embodiment, the digital therapeutic is administered over a period of eight weeks.

In one embodiment, the subject exhibits an EPDS score of less than 13 upon completion of the cognitive behavioral therapy content.

In one embodiment, the subject exhibits an improvement in Hamilton Rating Scale for Depression (HAMD) score upon completion of the cognitive behavioral therapy content.

In one embodiment, the method further includes administering a therapeutically effective amount of a pharmacotherapeutic to a subject to treat the perinatal mood disorders, wherein the digital therapeutic is administered in conjunction with the administration of the pharmacotherapeutic.

In one embodiment, the pharmacotherapeutic comprises an antidepressant, a depressant, an insomnia medication, an antipsychotic, or a combination thereof.

In some embodiments, there is provided a digital therapeutic configured to perform the methods described above.

In some embodiments, there is provided a digital therapeutic system configured to perform the methods described above.

In some embodiments, there is provided a computer system executing the digital therapeutic configured to perform the methods described above.

### BRIEF DESCRIPTION OF THE FIGURES

The accompanying drawings, which are incorporated in and form a part of the specification, illustrate embodiments of the invention and together with the written description serve to explain the principles, characteristics, and features of the invention. In the drawings:

FIG. 1 shows a diagram of an illustrative digital therapeutic system and systems for interacting therewith, in accordance with an embodiment of the present disclosure.

FIG. 2 shows a diagram of how prospective subjects were assessed and divided into intervention and control groups for a study of the digital therapeutic, in accordance with an embodiment of the present disclosure.

### DETAILED DESCRIPTION

This disclosure is not limited to the particular systems, devices and methods described, as these may vary. The terminology used in the description is for the purpose of describing the particular versions or embodiments only and is not intended to limit the scope of the disclosure.

### Definitions

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “pharmaceutical” is a reference to one or more pharmaceuticals and equivalents thereof known to those skilled in the art, and so forth.

As used herein, the term “about” means plus or minus 10% of the numerical value of the number with which it is being used. Therefore, about 50 days means in the range of 45 days to 55 days.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

As used herein, a “digital therapeutic” refers to software or content provided via software that is intended to treat or alleviate a disease, disorder, condition, or injury by generating and delivering a medical intervention that has a demonstrable positive therapeutic impact on a subject's health. A digital therapeutic could be delivered via a smartphone app, for example.

As used herein, “administering” when used in conjunction with a pharmacotherapeutic means to administer a therapeutic directly into or onto a target tissue or to administer a therapeutic to a subject whereby the therapeutic positively impacts the tissue to which it is targeted. “Administering” a composition may be accomplished by injection, topical administration, oral administration, buccal administration, sublingual administration, transdermal administration, or by any of these methods in combination with other known techniques. “Administering” can include administration by any individual, including, for example, self-administration or administration by a healthcare professional.

As used herein, “engaging” when used in conjunction with a digital therapeutic system, a cognitive behavioral therapy program, or other systems/programs that provide therapy content to subjects means to directly or indirectly interact and/or receive treatment or therapy from the system or program based on the provided therapy content. “Engaging” with such a system or program can be accomplished by reading therapy content, responding to therapy content (e.g., filling out questionnaires or answering questions), participating in a conversation with the therapy content (e.g., reading and answering material meant to simulate a conversational flow), and/or otherwise interacting with the provided therapy content. “Engaging” can include a user engaging with the therapy content or the user being instructed to engage with the content (e.g., by a healthcare provider (HCP)).

As used herein, “pharmacotherapy” or “pharmacotherapeutic” refers to a drug, medication, compound, or active pharmaceutical ingredient that is intended to case, inhibit, block, or reverse a disorder in a subject suffering from the disorder.

As used herein, a “therapeutically effective amount” or “effective amount” of a composition is a predetermined amount calculated to achieve the desired effect, i.e., to case, inhibit, block, or reverse a disorder. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. The compounds are effective over a wide dosage range. The compounds may be delivered once daily, once weekly, multiple times daily, or multiple times weekly. However, it will be understood that the effective amount administered will be determined by a physician in light of the relevant circumstances including the condition to be treated, the choice of compound to be administered, and the chosen route of administration, and therefore the above dosage ranges are not intended to limit the scope of embodiments herein in any way. A therapeutically effective amount of compound of this invention is typically an amount such that when it is administered in a physiologically tolerable excipient composition, it is sufficient to achieve an effective systemic concentration or local concentration in the tissue.

As used herein, a “subject” can refer to any individual that is making use of the digital therapeutic, the digital therapeutic system, and/or the digital therapy app. Subjects could include subjects that have been prescribed the digital therapeutic or other users that are utilizing the digital therapeutic without a prescription.

As used herein, the terms “perinatal mood disturbance” and “perinatal mood disorder” refer to mood and anxiety symptoms that occur during pregnancy or up to one year postpartum. Symptoms can include feeling sad or depressed, increased crying, diminished interest in becoming a mother, feelings of guilt (particularly around motherhood), strong feelings of anxiety and tension, difficulty sleeping, thoughts of self-harm or suicide, decreased energy, either loss of or increase in appetite, difficulty focusing, impaired memory, feeling restless or irritable, or suffering from headaches, chest pains, heart palpitations, numbness, or hyperventilation. Perinatal mood disorders could include, for example, PPD, postpartum anxiety disorders (e.g., panic disorder, obsessive compulsive disorder, or generalized anxiety disorder), and postpartum psychosis.

### Digital Therapeutic System

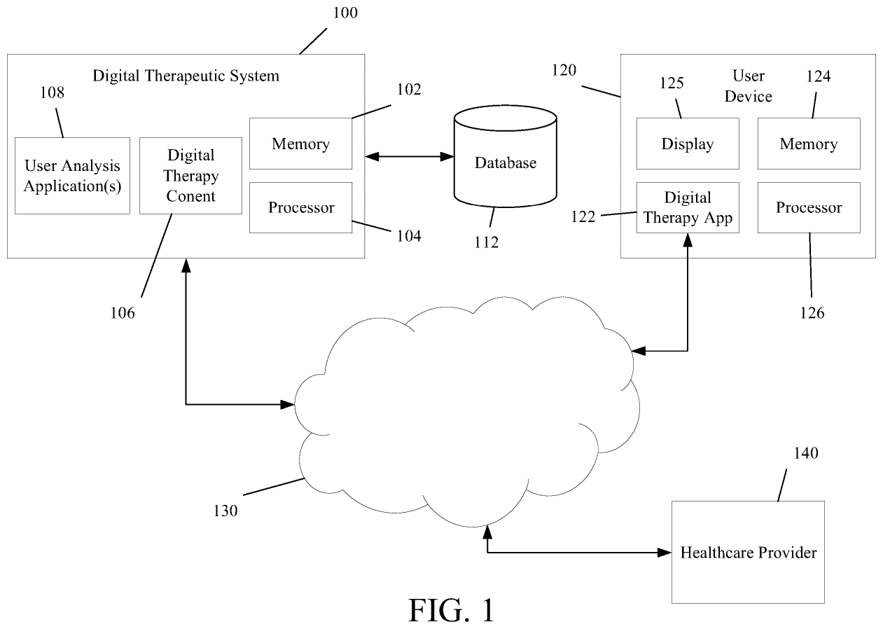

The present application is generally directed to the provision of digital therapeutic services to users. In one embodiment shown in FIG. 1, a digital therapeutic system 100 may be accessed via a user device 120 through a network 130 (e.g., the Internet or another telecommunication network). The digital therapeutic system 100 may provide digital therapy content 106 to a user through the device 120. The digital therapeutic system 100 may include a computer system, such as a server or server system, that is configured to provide the digital therapy content 106 to a user through the user device 120. The digital therapeutic system 100 may further include a memory 102 and a processor 104 that is adapted to execute instructions stored in the memory 102 to provide the digital therapy content 106 to the user device 120 and perform other tasks described herein. The user device 120 may include a mobile device (e.g., a smartphone), a tablet, a laptop, a desktop computer, or any other device that is able to access and/or display the digital therapy content 106. In one embodiment, the user may download a smartphone app 122 on the user device 120 through which the digital therapeutic system 100 can be accessed to provide the digital therapy content 106 thereto. In other embodiments, the digital therapeutic system 100 may be accessed via, for example, a website, a web application, or as a software as a service (SaaS) model. The digital therapy app 122 may provide a user interface through which the user can access, view, and/or interact with the digital therapy content 106 provided via the digital therapeutic system 100.

In some embodiments, the digital therapeutic system 100 can be communicably coupled to a user device 120 (e.g., a smartphone) and/or an HCP system 140 via a network 130. The digital therapeutic system 100 can be embodied as a cloud computing system (e.g., a distributed system) that provides the digital therapy content 106 to the user device 120, which can be accessed by the subject via the digital therapy app 122. In some embodiments, the digital therapeutic system 100 can further include or execute various additional algorithms or user applications 108 to analyze data received either directly or indirectly from the user in order to, for example, control or adjust the digital therapy content 106 provided to the subject. In some embodiments, the digital therapeutic system 100 can further be communicably coupled to an HCP system 140, which is described below. In some embodiments, the network 130 allows for interaction between subjects and/or HCPs 140 and the digital therapy content 106. For instance, the digital therapeutic system 100 may provide the subject with access to the digital therapy content 106 and receive user input or event data associated with the subject's interaction with the digital therapy content 106. In turn, the digital therapeutic system 100 may store the input and/or event data in a database 112.

In some embodiments, the digital therapeutic system 100 can be embodied as one or more servers that are programmed to deliver digital therapy content 106 to the user via the digital therapy app 122. In some embodiments, the digital therapeutic system 100 can be embodied as a cloud computing system. The cloud computing system can be a distributed system (e.g., remote environment) having scalable/elastic computing and storage resources. The cloud computing resources can facilitate communications with the user device 120 and store data in the database 112. In some examples, the database 112 can reside on a standalone computing device. The digital therapeutic system 100 can provide the user with the digital therapy content 106 that is delivered via the digital therapy app 122 executable on the user device 120.

The network 130 can include any type of network that allows sending and receiving communication signals, such as a wireless telecommunication network, a cellular telephone network, a time division multiple access (TDMA) network, a code division multiple access (CDMA) network, global system for mobile communications (GSM), a third generation (3G) network, fourth generation (4G) network, fifth generation (5G) network, a satellite communications network, and/or other communication networks. The network 130 can include one or more of a Wide Area Network (WAN), a Local Area Network (LAN), and a Personal Area Network (PAN). In some examples, the network 130 includes a combination of data networks, telecommunication networks, and a combination of data and telecommunication networks. The user device 120 and the digital therapeutic system 100 can communicate with each other by sending and receiving signals (wired or wireless) via the network 130. In some examples, the network 130 provides access to cloud computing resources, which can include elastic/on-demand computing and/or storage resources available over the network 130.

The user device 120 can include, but is not limited to, a portable electronic device (e.g., smartphone, cellular phone, personal digital assistant, personal computer, or wireless tablet device), a desktop computer, or any other electronic device capable of sending and receiving information via the network 130. The user device 120 includes data processing hardware (e.g., a processor 126), memory 124, and a display 125 in communication with the data processing hardware. In some examples, the user device 120 includes a keyboard (either a physical or a software-based keyboard), mouse, microphones, and/or a camera for allowing the user to input data. In addition to or in lieu of the display 125, the user device 120 can include one or more speakers to output audio data to the user. For instance, the digital therapy app 122 can be configured to provide audible alerts to the user or play audio content for the user. In some embodiments, the user device 120 can execute the digital therapy app 122 that the user has downloaded and is stored in the memory 124 of the user device 120 to establish a connection with the digital therapeutic system 100 and access the digital health content 106. In other embodiments, the digital therapy app 122 can include a web-based application through which the user can access the digital health content 106. In some embodiments, the user can have access to the digital health content 106 for a defined time period (e.g., 3 months). In some embodiments, the access to the digital health content 106 can be controlled based on whether the user has subscribed to the service provider controlling the digital therapeutic system 100. The digital therapy app 122, when executed by the data processing hardware of the user device 120, can be configured to display a variety of graphical user interfaces (GUIs) on the display 125 that, among other things, allows the user to interact with the digital therapy app 122.

In some embodiments, user devices 120 can access the digital therapeutic system 100 resources via a variety of different Infrastructure as a service (IaaS) standards, including, e.g., Amazon Elastic Compute Cloud (EC2), Open Cloud Computing Interface (OCCI), Cloud Infrastructure Management Interface (CIMI), or OpenStack standards. Some IaaS standards can allow clients access to resources over HTTP and can use Representational State Transfer (REST) protocol or Simple Object Access Protocol (SOAP). In some embodiments, user devices 120 can access the digital therapeutic system 100 resources via a variety of different Platform as a Service (PaaS) interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that are or can be built on REST, HTTP, XML, or other protocols. In some embodiments, user devices 120 can access digital therapeutic system 100 resources via a variety of different of web-based user interfaces (e.g., when the digital therapeutic system 100 is provided as a SaaS). In some embodiments, user devices 120 can also access SaaS resources through smartphone or tablet applications (e.g., Salesforce Sales Cloud or Google apps) or client operating systems.

In some embodiments, the digital therapeutic system 100 and/or the digital therapy app 122 can further be configured to transmit and/or receive information from a third-party system, such as an HCP system 140. For example, the digital therapeutic system 100 and/or the digital therapy app 122 could be configured to retrieve user medical information from an HCP (e.g., in the form of an electronic medical record) or provide notifications to the healthcare provider.

As one potential implementation, the digital therapeutic system 100 may be configured to provide digital therapeutic services associated with or otherwise related to perinatal mood disorders, such as postpartum depression. In one embodiment, the digital therapy content 106 may be designed to manage symptoms of depression and anxiety during pregnancy or after delivery, i.e., perinatal mood disorders. In particular, the digital therapy content 106 may be designed for developing skills to help manage symptoms of anxiety and depression, promoting social support and relationship quality, and encouraging help-seeking behavior in users. In one embodiment, the digital therapy content 106 provided via the digital therapy app 122 may encourage users to upload audio and/or video content, which may in turn be received by the digital therapeutic system 100. For example, the digital therapy content 106 may request that users upload video (e.g., video journal entries) and/or audio of themselves. The digital therapy content 106 may request that users upload the video and/or audio on a periodic (e.g., daily), a nonperiodic basis, or in response to various user inputs or other parameters. To facilitate the recording of the video and/or audio content, the user device 120 may include a camera, a microphone, and/or other recording devices.

The uploaded user video and/or audio may be stored in, e.g., a database 112 associated with the digital therapeutic system 100. In some embodiments, the digital therapeutic system 100 may be configured to customize the digital therapy content 106 delivered to the user, notify the user as to any detected trends, and/or notify healthcare professionals accordingly.

In one embodiment, the digital therapeutic system 100 may further be communicably connected to a healthcare provider 140 associated with the user. For example, the digital therapeutic system 100 may be configured to upload data to an electronic medical record (EMR) associated with the user, send a message (e.g., email) to the user's healthcare professional, or update a user profile provided by the digital therapeutic system 100 that is accessible by the healthcare provider 140. In one embodiment, the digital therapeutic system 100 may only notify the healthcare provider 140 in response to appropriate permissions being granted by the user.

In some embodiments, the digital therapeutic could be available without a prescription and could be freely accessible and downloadable by any users. In other embodiments, the digital therapeutic could require a prescription to access. For example, the ability to download the app and/or access the digital therapeutic content could require login credentials, a QR code, or other techniques for gating access to the digital therapeutic content on the digital therapeutic system 100. In various embodiments, the digital therapeutic 120 could be prescribed to the subject (e.g., by a healthcare provider 140). In some instances, the person using or being administered the digital therapeutic may be referred to as a “user.” A “user” may include a subject or any other person using or being administered the digital therapeutic 120, irrespective of whether the digital therapeutic 120 was prescribed to that person.

In some embodiments, the digital therapeutic 120 may include or be combined with traditional drug therapy, which similarly may or may not require a prescription. An authorized HCP 140 may prescribe the subject the digital therapeutic app 122 designed to treat symptoms in the subject. The HCP 140 may include a physician, nurse, clinician, or other qualified health professional. The HCP 140 may provide any suitable level of supervision to the subject, including little to no supervision.

The digital therapy content 106 may be designed to provide personalized self-help tools for women, such as women attempting to manage symptoms of depression and anxiety during pregnancy or after delivery. The digital therapy content 106 may guide expecting and new mothers through their journey, casing the transition to parenthood and providing helpful tips, self-guided strategies and reminders along the way. The digital therapy content 106 may be designed to be completed over a particular time period (e.g., 8 weeks). The digital therapy content 106 may include a series of modules focused on developing skills to help manage symptoms of anxiety and depression, promoting social support and relationship quality, and encouraging help-seeking behavior. Digital tools, such as provided by the digital therapy content 106, are useful in delivering self-guided personal development and treatment strategies due to their flexibility, privacy, personalization, and case-of-use. Further, the digital therapy content 106 may include interactive exercises that provide personalized feedback to support learning and built-in trackers make it easy for users to track their progress through the digital therapy content 106.

In some embodiments, the digital therapy content 106 may be designed to provide cognitive behavioral therapy (CBT) to users. CBT is a psychotherapeutic approach that is based on the concepts that (i) psychological problems are based, in part, on faulty or unhelpful ways of thinking, (ii) psychological problems are based, in part, on learned patterns of unhelpful behavior, and (iii) people suffering from psychological problems can learn better ways of coping with them, thereby relieving their symptoms and becoming more effective in their lives. CBT includes a number of psychological techniques intended to address the aforementioned concepts that share a theoretical basis in behavior learning theory and cognitive psychology. Accordingly, the digital therapy content 106 may include one or more modules providing CBT content that users can interact with or view to receive CBT. The digital therapy content 106 may, for example, by developed by or in concert with clinical psychologists and other experts to encourage development of skills to help manage the symptoms of depression and anxiety using CBT-based principles. The digital therapy content 106 can be designed to minimize the risk factors for postpartum depression, including lack of social support, along with the promotion of psychological processes and self-regulatory skills such as emotion regulation, psychological flexibility, and self-compassion.

Accordingly, the digital therapeutic system 100 can provide a remotely managed, fully automated digital therapy, which can be used by the subject without the involvement or guidance of a physician or therapist, although it may be beneficial to have the support of a healthcare provider for certain subject groups. The digital therapy content 106 can be tailored to individual subjects and is conveniently available whenever the user wishes to access the digital therapy content 106. The digital therapy content 106 may be adjusted based on input from the user, e.g., using artificial intelligence to analyze which content 106 the user is interacting with, the manner in which the user interacts with the content 106 and/or the digital therapy app 122, input from the user, and so on. Accordingly, the digital therapeutic system 100 can respond individually to a subject's or a consumer's feedback and adjust the content 106 accordingly.

### Treatment of Perinatal Mood Disorders Using Digital Therapeutics

Current treatments for individuals suffering from perinatal mood disorders can include psychotherapy (e.g., CBT or interpersonal therapy) and/or pharmacotherapy. However, many individuals do not receive the required level of care because access to psychotherapeutic medical services can be limited. Due to the unpredictably with which symptoms can manifest, individuals suffering from perinatal mood disorders and other mood disorders could benefit from psychotherapeutic assistance being immediately available in the form of a digital therapeutic that can be readily accessed, without the need to schedule time with a medical professional. Therefore, digital tools can be valuable in order to provide individuals suffering from perinatal mood disorders with the ability to both receive the therapy that they may not otherwise be able to access, as well as the ability to receive therapy when and where they need it with nothing more than their computer or smartphone. The digital therapeutics described herein can be provided as an adjunct to the standard of care for perinatal mood disorders, in combination with pharmaceutical treatments (i.e., pharmacotherapies), or as a standalone therapeutic. In one embodiment, the present disclosure is directed to a combination therapy, namely, treating subjects with a digital therapeutic configured to provide CBT to the subject in combination with a pharmaceutical intervention.

Described herein are various techniques, system, and/or therapeutics for treating perinatal mood disorders and other mood disorders using, at least in part, digital therapeutics. In particular, subjects can engage with the digital therapeutic and/or digital therapy content 106 in any manner that allows the subject to receive treatment from the digital therapeutic for the disorder or condition that is affecting the subject. In other words, subjects can engage with the digital therapeutic in a variety of different manners that are sufficient to allow the subject to receive the treatment therefrom. As noted above, in some embodiments, the digital therapeutic can be provided in the form of a smartphone app that is downloaded by the subject. In some embodiments, the digital therapeutic could be provided in the form of a website or web app that the subject can access via a web browser (e.g., Google Chrome, Mozilla Firefox, Internet Explorer or Safari). The digital therapeutic is configured to implement CBT techniques for treating subjects suffering from perinatal mood disorders, either as a standalone treatment or in combination with one or more pharmacotherapeutics. In some embodiments, the CBT content provided by the digital therapeutic can be configured to complement the administration of the pharmacotherapeutic.

In some embodiments, the digital therapy content 106 can be presented to the user via the digital therapy app 122 as a series of modules or lessons. The modules could present different types of information to the subject, including in different formats, such as text, audio, or video. In various embodiments, the modules could include content that is designed to be consumed passively (e.g., articles or videos), interactive content (e.g., surveys or quizzes), or any combination thereof. In some embodiments, the modules could implement different CBT techniques or otherwise include various types of CBT content designed to treat perinatal mood disorders. The modules implement CBT by discussing topics like automatic thoughts, alternative thinking, feeling overwhelmed, interpersonal relationships, and managing anxiety. For example, each module could explain the context or rationale of the therapeutic element and suggest ways that the subject could implement the CBT-based techniques into their day-to-day life to treat their perinatal mood disorders. In various embodiments, subjects could be expected to complete, for example, one module per day. In other embodiments, the digital therapy content 106 could be designed such that subjects are able to consume or interact with the digital therapy content 106 as desired. In some embodiments, the content within the modules, the order in which the modules are presented to the subject, which modules are presented to the subject, and/or the number of modules presented to the subject could be customized based on the manner in which the user interacts with the digital therapy content.

In some embodiments, the digital therapeutic treatment described herein can include or be combined with standard of care for perinatal mood disorders, such as a pharmacotherapy and/or psychotherapy, which similarly may or may not require a prescription. An authorized HCP 140 may prescribe the digital therapeutic described herein and/or the drug therapy to the subject. In some embodiments, the drug therapy could include antidepressants, depressants, insomnia medications, and/or antipsychotics. Antidepressants could include, for example, selective serotonin reuptake inhibitors (SSRIs) (e.g., sertraline, paroxetine, or fluoxetine) and serotonin-norepinephrine reuptake inhibitors (SNRIs) (e.g., duloxetine), bupropion, tricyclic antidepressants (TCAs) (e.g., amitriptyline or imipramine). Depressants could include, for example, benzodiazepines. In particular illustrative embodiments, the drug therapy taken by subjects in combination with the digital therapeutic could include sertraline (i.e., Zoloft), venlafaxine (i.e.,Effexor), brexanolone (i.e., Zulresso), paroxetine (i.e., Paxil), escitalopram (i.e., Lexapro), bupropion hydrochloride (i.e., Wellbutrin), citalopram (i.e., Celexa), aripiprazole (i.e., Abilify), or any combination thereof. In some embodiments, the systems and methods described herein can include administering a therapeutically effective amount of a pharmacotherapy in combination with administering the digital therapeutic to the subject.

In some embodiments, the digital therapeutic described herein is intended for use by various subject groups, particularly women. In some embodiments, the digital therapeutic can be intended for use by women that are at risk for perinatal mood disorders. In some embodiments, the digital therapeutic can be intended for use by women that have been diagnosed with one or more perinatal mood disorders. In some embodiments, the digital therapeutic can be intended for use by women that are 18 years of age or older. In some embodiments, the digital therapeutic can be intended for use by new mothers and those with previous births.

In some embodiments, the digital therapeutic described herein can be configured to be completed over a define time period. In some embodiments, the digital therapeutic can be considered to be completed when the subject was completed all of the modules that are provided to her. For example, the time period for completion may be, without limitation, two weeks, four weeks, six weeks, eight weeks, ten weeks, or twelve weeks. In one illustrative embodiment, the digital therapeutic is configured to be completed over a period of eight weeks.

In some embodiments, the digital therapeutic described herein can be configured to achieve particular subject endpoints. In one embodiment, the digital therapeutic can be configured to cause subjects to achieve a threshold score on the Edinburgh Postnatal Depression Scale (EPDS) or a threshold improvement in the subject's EPDS score. The EPDS is a screening tool that was developed to assist health professionals with detecting mothers suffering from perinatal mood disorders, including PPD. The EPDS is a questionnaire that consists of ten specifically defined statements generally asking how the subject has felt over the past seven days. For each statement, the subject checks off one of four possible answers that is closest to how she has felt during the past week. Responses are scored based on the seriousness of the symptom associated with each statement. The total score is found by adding together the scores for each of the ten statements. A total score of thirteen or more is generally considered to indicate that the individual is possibly experiencing depressive symptoms and may require follow-up. In one embodiment, the digital therapeutic can be configured to achieve an improvement of four or more points on the EPDS in subjects that have completed the digital therapeutic, as compared to a control group, i.e., individuals who have not completed the digital therapeutic. In another embodiment, the digital therapeutic can be configured to reduce a subject's EPDS score below 13, the commonly accepted threshold of PPD. In another embodiment, the digital therapeutic could be configured to achieve an improvement in Hamilton Rating Scale for Depression (HAMD) scores for subjects. The study described below determined that improvements in HAMD scores were aligned with improvements in EPDS scores for subjects.

Accordingly, the digital therapeutic described herein can be used to treat subjects that are affected by perinatal mood disorders, either as a standalone treatment or in combination with a pharmacotherapy. The digital therapeutic treats the symptoms associated with perinatal mood disorders, thereby improving patient outcomes. In particular, the digital therapeutic can be configured to produce marked improvement in subjects' wellbeing, as measured by certain endpoints, such as EPDS score.

### Illustrative Clinical Trial Protocol & Results

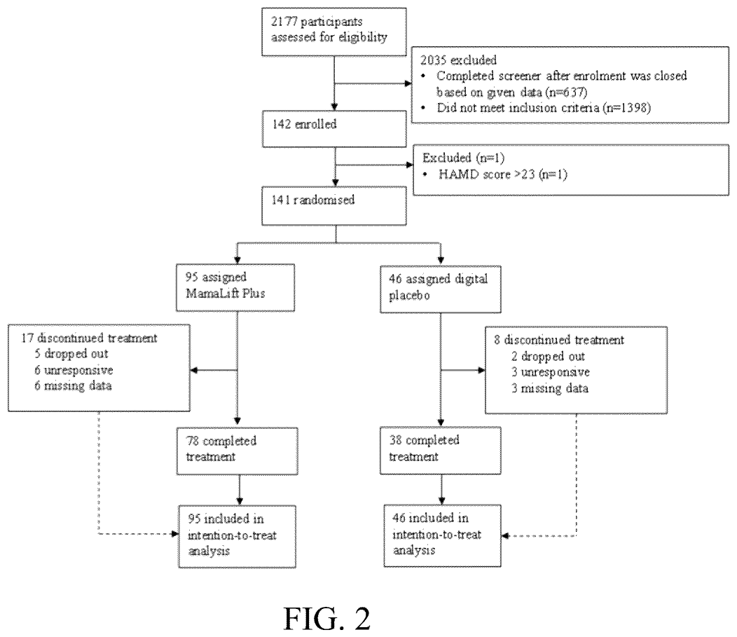

In order to evaluate the digital therapeutic described herein, a national, randomized, placebo-controlled pivotal trial was conducted for eight weeks in 141 subjects. The subjects were randomized in a 2:1 ratio into an intervention group (N=95) that was administered the digital therapeutic and a control group (N=46) that was provided a placebo. FIG. 2 illustrates how prospective subjects were assessed for eligibility and divided into the intervention and control (placebo) groups, as well as the number of subjects in each group that completed the treatment. The control device delivers information on a variety of topics related to mother and infant wellbeing. The subjects inducted into the study had baseline EPDS scores from 13 to 19, and a confirmed diagnosis of PPD prior to enrollment.

This trial was a randomized study comparing a digital therapy in accordance with the embodiments described above in combination with treatment as usual (TAU) to a digital app matched placebo in combination with TAU conducted in participants diagnosed with PPD. In particular, the main objective of the study was to evaluate the clinical efficacy of the digital therapeutic intervention described herein to a placebo digital app in participants with EPDS scores from 13 to 19, inclusive, and a clinical diagnosis of PPD for a period of nine calendar weeks. The study population consisted of women from 18 to 50 years of age who have had a live birth within 3 months prior to study start date and have mild to moderate PPD. Individuals who were diagnosed with Serious Mental Illnesses (SMIs), as defined by psychotic disorders, bipolar disorders, and either major depression with psychotic symptoms or treatment-resistant depression; severe depression (which includes participants hospitalized for depression or currently using more than 2 medications for depression at the same time); or cognitive impairments were excluded from the study.

The study obtained a range of data on each patient, including depression or anxiety (e.g., based on EPDS or HAMD assessments), other mental health and quality of life indicators, user data collected via the digital therapeutic app (in the intervention group only), utilization of healthcare (including office or outpatient visits, emergency room visits, hospitalizations, behavioral health services, and telehealth services), and adherence to app use and user engagement (in the intervention group only). Other mental health indicators included a survey of mental health treatment received by the subjects and prescribed medication being taken by the subjects. The user data collected via the app included daily mood, sleep data, learning activities, and daily activities. Subjects' depression severity was assessed via the EPDS at screening, an interim point (week 4), and study completion (week 9). HAMD assessments were conducted by a behavioral health professional to assess symptoms of depression and anxiety as reported by the participant at the clinical assessment phase and at the end of the study period as an exploratory endpoint.

Participants in both groups were prompted or able on a daily basis to enter information on their mobile apps (i.e., the digital therapeutic described herein for the intervention group and the placebo app for the control group). Responses to activities associated with Cognitive Behavioral Therapy (CBT), Interpersonal Therapy (IPT), Dialectical Behavioral Therapy (DBT) and Behavior Activation Therapy (BAT) collected in the intervention group were assessed on a daily basis to monitor for potential adverse events reported by the participant. For participants in the digital placebo (i.e., control) group, the subjects' responses to daily questions and any free form text entered were assessed to monitor for potential adverse events reported by the subject. If an adverse event was reported or identified for any subject, a notification was provided to the subject informing them to seek immediate help. Additionally, the incident was reported to the study team to evaluate and notify the medical monitor for follow-up (if needed). At week 9 (or end of study participation), a licensed behavioral health therapist or medical professional reassessed symptoms associated with depression reported by each participant using the HAMD-21.

In the study, the digital therapeutic in accordance with the digital therapy systems described herein, which was provided to the intervention group, utilized personalized cognitive restructuring as the primary therapeutic component to improve the symptoms of PPD. This cognitive restructuring is mapped onto standard, evidence-based CBT techniques that were initially developed for and provided in a face-to-face context. Content provided by the digital therapeutic app was conveyed to patients via a sequence of eight self-guided and interactive treatment modules designed to parallel the traditional, face-to-face delivery and structure of CBT sessions (e.g., weekly over a period of eight to nine weeks sequentially). Subjects were strongly encouraged to complete all eight modules in the digital therapy application at the rate of one module per week, but the entire program lasted up to 9 weeks with the baseline and posttreatment assessment periods. Full engagement by prescribing clinicians and their support staff was used to greatly facilitate effective use by subjects of the prescribed digital therapeutic and their continued use in conjunction with ongoing monitoring by the clinician.

The key modular content areas demonstrated efficacy in the treatment and management of PPD. The digital therapy content also included recent developments in acceptance and commitment-based therapies designed specifically for the perinatal context. The Beck Institute, pioneers in the area of CBT, has indicated that patient symptoms can improve from using CBT therapies after 8-10 sessions, depending on client goals and individual case formulation. Intervention content was delivered through a variety of features, including text, personalized goal-setting, graphical feedback based on inputted symptoms, animations and illustrations to enrich comprehension, quizzes to test and enhance user knowledge, video vignettes to promote user identification with material, and video-based expert explanations. The digital therapy app also sent “nudges” via SMS to encourage users to visit the app daily.

Only a limited sample of women took medications during the study. 39.3% of subjects in the intervention group reported taking some prescribed medication at baseline. 36.4% of subjects in the control group reported taking some prescribed medication at baseline. 25% of subjects in the intervention group reported taking some medication at end of treatment. 26.3% of subjects in the control group reported taking some medication at end of treatment.

Additional features of the digital therapeutic app included a daily tracker in which patients were able to self-monitor and record standardized sleep parameters (e.g., sleep and naps), self-reports of perceived sleep quality, and self-reports of perceived energy level. In addition, the digital therapy app offered a daily mood tracker and activity tracker. The digital therapy app also provided personalized cognitive restructuring guidance based on the individual's beliefs, context, and attitudes.

For eight weeks, subjects were provided with both psychoeducational content and therapeutic strategies on a weekly basis. Subjects were encouraged to spend 8-12 minutes per day on the app to achieve the intended therapeutic “dosage.” During that time, they were instructed to visit several key functions of the app, including the daily learning section, mood tracker, sleep tracker, and activity tracker. The daily learning section included brief content that address key topics for the management of PPD. The mood tracker allowed users to monitor their moods. The sleep tracker helped users understand their sleep patterns. The activity tracker enabled users to track their pleasant activities and activities of daily living. Content contained in these trackers enabled self-care that supports positive well-being and also helps users understand the interplay between mood, sleep, and time spent doing things they enjoy. In addition to these core functions of the digital therapeutic platform, subjects were encouraged to visit the digital therapeutic platform resource library of care concerns where they could find assistance on various topics of interest to mothers of newborns.

To facilitate its use as an adjunct to standard care under the supervision of a qualified health care provider, the digital therapeutic system includes a clinician dashboard. The dashboard summarizes use of the mobile application by the subject, thereby enabling the treating clinician to assess and monitor their patient's progress through the therapeutic materials during the prescription period. Clinical data collected via the patient interface, including self-reports of depressive symptoms and/or moods and sleep data, are also displayed via the clinician dashboard. All data are encrypted and compliant with data privacy and patient confidentiality requirements of the Health Insurance Portability and Accountability Act. These features are intended to support the clinician with patient follow up and clinical decision making by communicating information about therapeutic adherence and patient-reported outcomes that are critical to evaluating the effectiveness of the digital therapeutic.

The placebo app included no branding or images. Specifically, the participant did not receive CBT content; however, they did receive limited daily interactive content. In addition, weblinks for content related to PPD or disease-state symptomatology were provided on a daily basis.

End points for this study included (i) the proportion of women that improve EPDS by >=4 points at their end of study assessment, (ii) the proportion of women that improve EPDS to <13 points at their end of study assessment, and (iii) the improvement in HAMD score at end of study. The trial met its primary endpoint, which was a greater than 4-point improvement in the EPDS score of the subjects that were administered the digital therapeutic. The EPDS is the most commonly used depression screening tool for the perinatal population and an improvement of four or more points is clinically meaningful. In particular, approximately 83% of participants in the intervention group achieved a ≥4-point improvement in EPDS score, compared to only 22% in the control group (p-value<0.001). Approximately 82% of subjects in the intervention group achieved an EPDS reduction to <13, compared to only 30% in the control arm (p-value<0.001). This substantial difference between subjects who scored less than 13 points on the EPDS is significant because, as noted above, subjects with EPDS scores ≥13 are candidates for interventional therapy.

Accordingly, the study demonstrates the efficacy of the digital therapeutic to reduce symptoms of depression in women with PPD or other perinatal mood disorders. Further, the digital therapeutic described herein improves treatment options for subjects suffering from perinatal mood disorders by delivering evidence-based CBT to reduce symptoms of depression.

### Embodiments

Embodiment 1. A method for treating perinatal mood disorders in a subject diagnosed with or at risk for the perinatal mood disorders, the method comprising: administering a digital therapeutic to the subject, the digital therapeutic configured to provide cognitive behavioral therapy content to the subject to treat the perinatal mood disorders; wherein the perinatal mood disorders are treated, thereby causing the subject to exhibit an improvement of four or more points on the Edinburgh Postnatal Depression Scale (EPDS) upon completion of the cognitive behavioral therapy content relative to a control subject that was not administered the digital therapeutic.

Embodiment 2. The method of Embodiment 1, wherein the perinatal mood disorders comprise postpartum depression.

Embodiment 3. The method of Embodiment 1 or Embodiment 2, wherein the digital therapeutic comprises a plurality of modules.

Embodiment 4. The method of any one of Embodiments 1-3, wherein the digital therapeutic is administered over a period of eight weeks.

Embodiment 5. The method of any one of Embodiments 1-4, wherein the subject exhibits an EPDS score of less than 13 upon completion of the cognitive behavioral therapy content.

Embodiment 6. The method of any one of Embodiments 1-5, wherein the subject exhibits an improvement in Hamilton Rating Scale for Depression score upon completion of the cognitive behavioral therapy content.

Embodiment 7. The method of any one of Embodiments 1-6, further comprising: administering a therapeutically effective amount of a pharmacotherapeutic to a subject to treat the perinatal mood disorders, wherein the digital therapeutic is administered in conjunction with the administration of the pharmacotherapeutic.

Embodiment 8. The method of Embodiment 7, wherein the pharmacotherapeutic comprises an antidepressant, a depressant, an insomnia medication, an antipsychotic, or a combination thereof.

Embodiment 9. A digital therapeutic for treating perinatal mood disorders in a subject diagnosed with or at risk for the perinatal mood disorders, the digital therapeutic comprising instructions configured to be stored in a memory and executed by a processor that, when executed by the processor, cause the digital therapeutic to provide cognitive behavioral therapy content to the subject to treat the perinatal mood disorders, wherein the perinatal mood disorders are treated, thereby causing the subject to exhibit an improvement of four or more points on the Edinburgh Postnatal Depression Scale (EPDS) upon completion of the cognitive behavioral therapy content relative to a control subject that was not administered the digital therapeutic.

Embodiment 10. The digital therapeutic of Embodiment 9, wherein the perinatal mood disorders comprise postpartum depression.

Embodiment 11. The digital therapeutic of Embodiment 9 or claim 10, wherein the digital therapeutic comprises a plurality of modules.

Embodiment 12. The digital therapeutic of any one of Embodiments 9-11, wherein the digital therapeutic is administered over a period of eight weeks.

Embodiment 13. The digital therapeutic of any one of Embodiments 9-12, wherein the subject exhibits an EPDS score of less than 13 upon completion of the cognitive behavioral therapy content.

Embodiment 14. The digital therapeutic of any one of Embodiments 9-13, wherein the subject exhibits an improvement in Hamilton Rating Scale for Depression score upon completion of the cognitive behavioral therapy content.

Embodiment 15. The digital therapeutic of any one of Embodiments 9-14, further comprising: administering a therapeutically effective amount of a pharmacotherapeutic to a subject to treat the perinatal mood disorders, wherein the digital therapeutic is administered in conjunction with the administration of the pharmacotherapeutic.

Embodiment 16. The digital therapeutic of Embodiment 15, wherein the pharmacotherapeutic comprises an antidepressant, a depressant, an insomnia medication, an antipsychotic, or a combination thereof.

Embodiment 17. A method for treating perinatal mood disorders in a subject diagnosed with or at risk for the perinatal mood disorders, the method comprising: administering a digital therapeutic to the subject, the digital therapeutic configured to provide cognitive behavioral therapy content to the subject to treat the perinatal mood disorders; wherein the perinatal mood disorders are treated, thereby causing the subject to exhibit an Edinburgh Postnatal Depression Scale score of less than 13 upon completion of the cognitive behavioral therapy content relative to a control subject that was not administered the digital therapeutic.

Embodiment 18. A method for treating perinatal mood disorders in a subject diagnosed with or at risk for the perinatal mood disorders, the method comprising: administering a digital therapeutic to the subject, the digital therapeutic configured to provide cognitive behavioral therapy content to the subject to treat the perinatal mood disorders; wherein the perinatal mood disorders are treated, thereby causing the subject to exhibit an improved score on the Hamilton Rating Scale for Depression upon completion of the cognitive behavioral therapy content relative to a control subject that was not administered the digital therapeutic.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art, all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., +10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

The functions and process steps herein may be performed automatically or wholly or partially in response to user command. An activity (including a step) performed automatically is performed in response to one or more executable instructions or device operation without user direct initiation of the activity.

1. A method for treating perinatal mood disorders in a subject diagnosed with or at risk for the perinatal mood disorders, the method comprising:
administering a digital therapeutic to the subject, the digital therapeutic configured to provide cognitive behavioral therapy content to the subject to treat the perinatal mood disorders;
wherein the perinatal mood disorders are treated, thereby causing the subject to exhibit an improvement of four or more points on the Edinburgh Postnatal Depression Scale (EPDS) upon completion of the cognitive behavioral therapy content relative to a control subject that was not administered the digital therapeutic.

2. The method of claim 1, wherein the perinatal mood disorders comprise postpartum depression.

3. The method of claim 1, wherein the digital therapeutic comprises a plurality of modules.

4. The method of claim 1, wherein the digital therapeutic is administered over a period of eight weeks.

5. The method of claim 1, wherein the subject exhibits an EPDS score of less than 13 upon completion of the cognitive behavioral therapy content.

6. The method of claim 1, wherein the subject exhibits an improvement in Hamilton Rating Scale for Depression score upon completion of the cognitive behavioral therapy content.

7. The method of claim 1, further comprising:
administering a therapeutically effective amount of a pharmacotherapeutic to a subject to treat the perinatal mood disorders, wherein the digital therapeutic is administered in conjunction with the administration of the pharmacotherapeutic.

8. The method of claim 7, wherein the pharmacotherapeutic comprises an antidepressant, a depressant, an insomnia medication, an antipsychotic, or a combination thereof.

9. A digital therapeutic for treating perinatal mood disorders in a subject diagnosed with or at risk for the perinatal mood disorders, the digital therapeutic comprising instructions configured to be stored in a memory and executed by a processor that, when executed by the processor, cause the digital therapeutic to provide cognitive behavioral therapy content to the subject to treat the perinatal mood disorders, wherein the perinatal mood disorders are treated, thereby causing the subject to exhibit an improvement of four or more points on the Edinburgh Postnatal Depression Scale (EPDS) upon completion of the cognitive behavioral therapy content relative to a control subject that was not administered the digital therapeutic.

10. The digital therapeutic of claim 9, wherein the perinatal mood disorders comprise postpartum depression.

11. The digital therapeutic of claim 9, wherein the digital therapeutic comprises a plurality of modules.

12. The digital therapeutic of claim 9, wherein the digital therapeutic is administered over a period of eight weeks.

13. The digital therapeutic of claim 9, wherein the subject exhibits an EPDS score of less than 13 upon completion of the cognitive behavioral therapy content.

14. The digital therapeutic of claim 9, wherein the subject exhibits an improvement in Hamilton Rating Scale for Depression score upon completion of the cognitive behavioral therapy content.

15. The digital therapeutic of claim 9, further comprising:
administering a therapeutically effective amount of a pharmacotherapeutic to a subject to treat the perinatal mood disorders, wherein the digital therapeutic is administered in conjunction with the administration of the pharmacotherapeutic.

16. The digital therapeutic of claim 15, wherein the pharmacotherapeutic comprises an antidepressant, a depressant, an insomnia medication, an antipsychotic, or a combination thereof.

17. A method for treating perinatal mood disorders in a subject diagnosed with or at risk for the perinatal mood disorders, the method comprising:
administering a digital therapeutic to the subject, the digital therapeutic configured to provide cognitive behavioral therapy content to the subject to treat the perinatal mood disorders;
wherein the perinatal mood disorders are treated, thereby causing the subject to exhibit an Edinburgh Postnatal Depression Scale score of less than 13 upon completion of the cognitive behavioral therapy content relative to a control subject that was not administered the digital therapeutic.

18. A method for treating perinatal mood disorders in a subject diagnosed with or at risk for the perinatal mood disorders, the method comprising:
administering a digital therapeutic to the subject, the digital therapeutic configured to provide cognitive behavioral therapy content to the subject to treat the perinatal mood disorders;
wherein the perinatal mood disorders are treated, thereby causing the subject to exhibit an improved score on the Hamilton Rating Scale for Depression upon completion of the cognitive behavioral therapy content relative to a control subject that was not administered the digital therapeutic.
